(19) United States
(12) Patent Application Publication
(10) Pub. No.: US 2012/0130084 A1
(43) Pub. Date: May 24, 2012

(54) PREPARATION OF FIPAMEZOLE

(75) Inventors: Ranjan Bhowmik Dipal, East Medinapore (IN); Lingam Yedugani, Ranga Reddy (IN); Raghavndra Rao Kamaraju, Secunderabad (IN); Subba Rao Jammula, Hyderabad (IN); Swapna Manikonda, Hyderabad (IN); Vilas H. Dahanukar, Hyderabad (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/362,211

(22) Filed: Jan. 31, 2012

Related U.S. Application Data
(63) Continuation of application No. PCT/US2010/043643, filed on Jul. 29, 2010.
(60) Provisional application No. 61/293,373, filed on Jan. 8, 2010.

(30) Foreign Application Priority Data
Jul. 31, 2009 (IN) 1814/CHE/2009

(51) Int. Cl. C07D 233/64; C07C 49/567; C07C 45/48

(52) U.S. Cl. 548/345.1; 568/314

(57) ABSTRACT

The application relates to processes for preparing fipamezole and its pharmaceutically acceptable salts, and intermediates thereof. It also provides intermediate compounds of Formula III and Formula IV, and processes for their preparation.

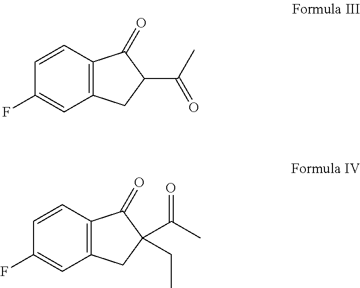

In connection with the filing of the above-identified application, which is a continuation of International Application No. PCT/US2010/043643 that was filed on Jul. 29, 2010, entry of the amendment presented herein is requested.

### INTRODUCTION

Aspects of the present patent application relate to processes for preparing fipamezole, its pharmaceutically acceptable salts, and intermediates thereof. In particular aspects it relates to processes for preparing fipamezole hydrochloride and to intermediates used in the processes.

The drug compound having the adopted name “fipamezole” has chemical names: 4-(2-ethyl-5-fluoro-2,3-dihydro-1H-inden-2-yl)-1H-imidazole; or 4-[(2 RS)-2-ethyl-5-fluoroindan-2-yl]-1H-imidazole; and is represented structurally by Formula I.

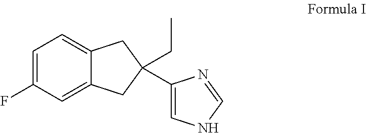

Fipamezole is a highly selective and long-acting adrenergic alpha-2 receptor antagonist and is currently undergoing clinical trials to treat symptoms of advanced Parkinson's disease such as dyskinesias, motor fluctuations and cognitive impairment. It has a good oral bioavailability and is useful in the treatment of cognitive disorders.

U.S. Pat. No. 5,498,623 discloses substituted imidazole derivatives including fipamezole and pharmaceutically acceptable salts thereof. It also discloses a process for the preparation of fipamezole involving nitration of 4-(2-ethyl-2,3-dihydro-1H-inden-2-yl)-1H-imidazole with urea nitrate and concentrated sulfuric acid; reduction of the resulting nitro compound to an amino compound using PtO₂; and reacting the amino compound with fluoroboric acid and sodium nitrite to obtain fipamezole, which is then converted to its hydrochloride salt. International Application Publication No. WO 04/063168 A1 discloses a process for preparing fipamezole, in which an alkali metal thiocyanate is used for the formation of an imidazole ring.

There is a continuing need for new and improved processes for the preparation of fipamezole with high purity and yield.

### SUMMARY

In an aspect, the present application relates to processes for the preparation of fipamezole or salts thereof, embodiments comprising:

a) reacting 5-fluoro-1-indanone of Formula II with a lower alkyl ester reagent, in the presence of a base, to form 1-(5-fluoro-1-oxo-2,3-dihydro-1H-2-indenyl)-1-ethanone of Formula III;

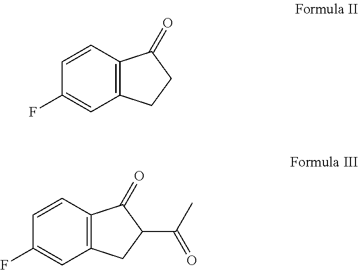

b) reacting 1-(5-fluoro-1-oxo-2,3,dihydro-1H-2-indenyl)-1-ethanone of Formula Ill with an ethyl halide, in the presence of a base, to form 1-(2-ethyl-5-fluoro-1-oxo-2,3,dihydro-1H-2-indenyl)-1-ethanone of Formula IV;

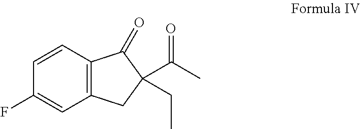

c) reducing 1-(2-ethyl-5-fluoro-1-oxo-2,3,dihydro-1H-2-indenyl)-1-ethanone of Formula IV with a reducing reagent to form 1-(2-ethyl-5-fluoro-2,3,dihydro-1H-2-indenyl)-1-ethanone of Formula V;

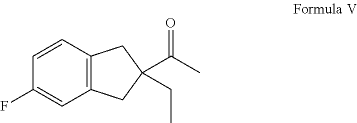

d) reacting 1-(2-ethyl-5-fluoro-2,3,dihydro-1H-2-indenyl)-1-ethanone of Formula V with a brominating agent to form 2-bromo-1-(2-ethyl-5-fluoro-2,3,dihydro-1H-2-indenyl)-1-ethanone of Formula VI; and

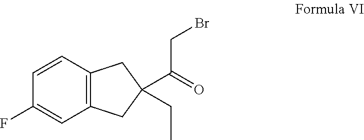

e) converting 2-bromo-1-(2-ethyl-5-fluoro-2,3,dihydro-1H-2-indenyl)-1-ethanone into fipamezole or a salt thereof, using formamide, optionally in the presence of ammonia.

In this multi-step process, each step is contemplated separately or in combinations of two or more steps for the preparation of fipamezole.

In an aspect, the present application provides processes for purifying fipamezole or a salt thereof, comprising:

a) providing a mixture of fipamezole or a salt thereof in an alcohol solvent;

b) combining the solution with a ketone anti-solvent and cooling to form a precipitate; and

c) recovering purified fipamezole or a salt thereof.

In an aspect the present application provides the intermediate compounds 1-(5-fluoro-1-oxo-2,3-dihydro-1H-2-indenyl)-1-ethanone of Formula III, and 1-(2-ethyl-5-fluoro-1-oxo-2,3,dihydro-1H-2-indenyl)-1-ethanone of Formula IV, and their uses in the preparation of fipamezole or a salt thereof.

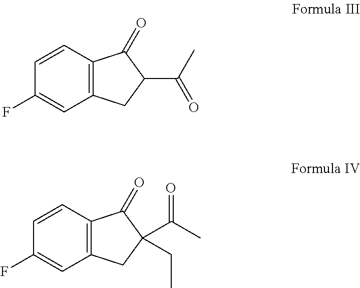

An aspect of the invention provides a process for preparing fipamezole or a salt thereof, comprising:

a) reacting 1-(2-ethyl-5-fluoro-1-oxo-2,3,dihydro-1H-2-indenyl)-1-ethanone of Formula IV with a reducing reagent, to form 1-(2-ethyl-5-fluoro-2,3,dihydro-1H-2-indenyl)-1-ethanone of Formula V;

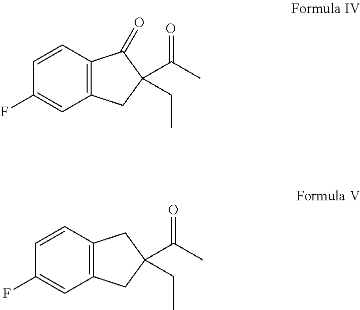

b) reacting 1-(2-ethyl-5-fluoro-2,3,dihydro-1H-2-indenyl)-1-ethanone of Formula V with a brominating agent, to form 2-bromo-1-(2-ethyl-5-fluoro-2,3,dihydro-1H-2-indenyl)-1-ethanone of Formula VI; and

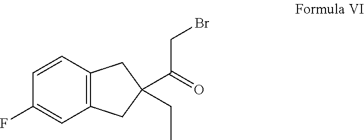

c) converting 2-bromo-1-(2-ethyl-5-fluoro-2,3,dihydro-1H-2-indenyl)-1-ethanone into fipamezole or a salt thereof.

An aspect of the invention provides a process for the preparation of 1-(2-ethyl-5-fluoro-1-oxo-2,3,dihydro-1H-2-indenyl)-1-ethanone of Formula IV, comprising:

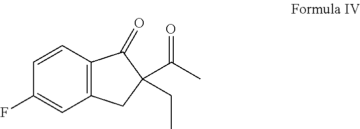

a) reacting 5-fluoro-1-indanone of Formula II with a lower alkyl ester reagent, in the presence of a base, to form 1-(5-fluoro-1-oxo-2,3-dihydro-1H-2-indenyl)-1-ethanone of Formula III; and

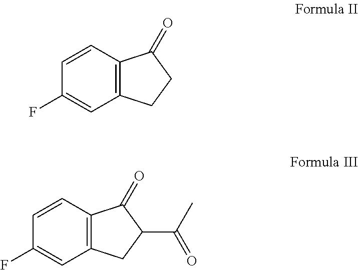

b) reacting 1-(5-fluoro-1-oxo-2,3,dihydro-1H-2-indenyl)-1-ethanone of Formula III with an ethyl halide, in the presence of a base, to form 1-(2-ethyl-5-fluoro-1-oxo-2,3,dihydro-1H-2-indenyl)-1-ethanone of Formula IV.

An aspect of the invention provides a compound 1-(5-fluoro-1-oxo-2,3-dihydro-1H-2-indenyl)-1-ethanone of Formula III.

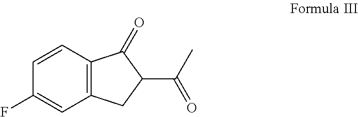

An aspect of the invention provides a compound 1-(2-ethyl-5-fluoro-1-oxo-2,3,dihydro-1H-2-indenyl)-1-ethanone of Formula IV.

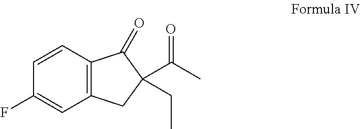

### DETAILED DESCRIPTION

In an aspect, the present application relates to processes for the preparation of fipamezole or a salt thereof, embodiments comprising:

a) reacting 5-fluoro-1-indanone of Formula II with a lower alkyl ester reagent, in the presence of a base, to form 1-(5-fluoro-1-oxo-2,3-dihydro-1H-2-indenyl)-1-ethanone of Formula III;

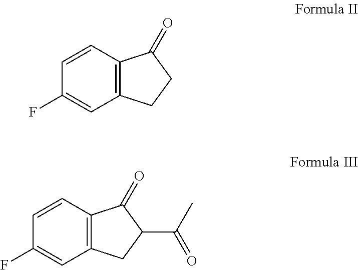

b) reacting 1-(5-fluoro-1-oxo-2,3,dihydro-1H-2-indenyl)-1-ethanone of Formula III with an ethyl halide, in the presence of a base, to form 1-(2-ethyl-5-fluoro-1-oxo-2,3,dihydro-1H-2-indenyl)-1-ethanone of Formula IV;

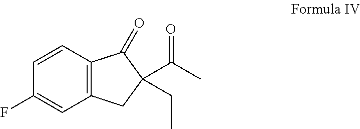

c) reducing 1-(2-ethyl-5-fluoro-1-oxo-2,3,dihydro-1H-2-indenyl)-1-ethanone of Formula IV with a reducing reagent to form 1-(2-ethyl-5-fluoro-2,3,dihydro-1H-2-indenyl)-1-ethanone of Formula V;

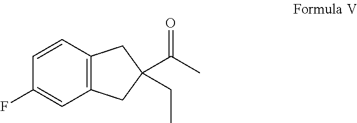

d) reacting 1-(2-ethyl-5-fluoro-2,3,dihydro-1H-2-indenyl)-1-ethanone of Formula V with a brominating agent to form 2-bromo-1-(2-ethyl-5-fluoro-2,3,dihydro-1H-2-indenyl)-1-ethanone of Formula VI; and

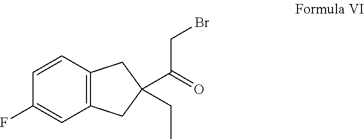

e) converting 2-bromo-1-(2-ethyl-5-fluoro-2,3,dihydro-1H-2-indenyl)-1-ethanone into fipamezole or a salt thereof, using formamide, optionally in the presence of ammonia.

Step a) involves a reaction of 5-fluoro-1-indanone of Formula II with a lower alkyl ester reagent, in the presence of a base, to form 1-(5-fluoro-1-oxo-2,3-dihydro-1H-2-indenyl)-1-ethanone of Formula III.

5-fluoro-1-indanone can be subjected to a Claisen condensation with a lower alkyl acetate in the presence of a base, to yield 2-acetyl-5-fluoro-indane-1-one.

Suitable lower alkyl esters that may be used in step a) include, without limitation thereto, ethyl acetate, n-propyl acetate, isopropyl acetate, n-butyl acetate, t-butyl acetate, and like.

Suitable bases that may be used in the reaction include, but are not limited to, sodium hydride, potassium hydride, sodium methoxide, sodium amide, and the like.

The reaction may be carried out in the presence of an inert solvent such as tetrahodrofuran (THF), hydrocarbons, such as, for example, toluene, xylene and the like; and mixtures thereof; or the alkyl ester may be used as the solvent.

Suitably, the reaction may be carried out at temperatures ranging from about 20 to 70° C., or about 50 to 55° C.

It has been observed that a C-alkylated indanone is isolated exclusively with more than 75% yield, and substantially no O-alkylated product is observed in this reaction.

Step b) involves a reaction of 1-(5-fluoro-1-oxo-2,3,dihydro-1H-2-indenyl)-1-ethanone of Formula III with an ethyl halide, in the presence of a base, to form 1-(2-ethyl-5-fluoro-1-oxo-2,3,dihydro-1H-2-indenyl)-1-ethanone of Formula IV.

Suitable ethyl halides that may be used include ethyl fluoride, ethyl chloride, ethyl bromide, and ethyl iodide.

Suitable bases that may be used include, but are not limited to: alkali metal hydroxides such as lithium hydroxide, sodium hydroxide, potassium hydroxide, and cesium hydroxide; alkaline metal hydroxides such as aluminum hydroxide, magnesium hydroxide, calcium hydroxide, and the like; alkali metal carbonates such as sodium carbonate, potassium carbonate, lithium carbonate, cesium carbonate, and the like; alkaline earth metal carbonates such as magnesium carbonate, calcium carbonate, and the like; and alkali metal bicarbonates such as sodium bicarbonate, potassium bicarbonate, and the like;

Suitable solvents that may be used in the reaction of step b) include, but are not limited to: polar aprotic solvents such as N,N-dimethylformamide, N,N-dimethylacetamide, N-methylpyrrolidone, pyridine, dimethylsulphoxide, sulpholane, formamide, acetamide, propanamide, and the like; tetrahydrofuran (THF); hydrocarbons, such as, for example, toluene and xylene; halogenated hydrocarbons, such as, for example, dichloromethane, dichloroethane, chloroform, chlorobenzene, and the like; and any mixtures thereof.

Suitably, the reaction may be carried out at temperatures ranging from about 20-70° C., or about 50-55° C.

After completion of the reaction, the reaction may be quenched by adding water slowly and then acidified with a mineral acid such as HCl. The product is extracted with a water immiscible organic solvent and the solvent is distilled to obtain the product as a residue.

Step c) involves reducing 1-(2-ethyl-5-fluoro-1-oxo-2,3,dihydro-1H-2-indenyl)-1-ethanone of Formula IV with a reducing agent to form 1-(2-ethyl-5-fluoro-2,3,dihydro-1H-2-indenyl)-1-ethanone of Formula V.

The removal of the benzylic ketone of the halogenated aryl 1,3-diketone can be achieved by a chemo-selective hydrogenation reaction, carried out in a polar solvent in the presence of an acid.

Suitable reducing agents that may be used in the reaction of step c) include, but are not limited to, metal catalysts, such as, for example, nickel, platinum, palladium, iridium, ruthenium, and the like, in combination with hydrogen. These metal catalysts can be used in concentrations about 2-20% w/w, or 5-10% w/w, on an inert solid support such as charcoal or alumina.

Suitable solvents that may be used in the reaction of step c) include, but are not limited to: C₁-₆ straight chain or branched alcohols, such as, for example, methanol, ethanol, isopropanol, butanol, and the like; hydrocarbons, such as, for example, toluene, xylene, and the like; and any mixtures thereof.

The reaction may be carried out in the presence of an acid, such as, for example, hydrochloric acid, hydrobromic acid, sulphuric acid, phosphoric acid, acetic acid, and the like.

After completion of the reaction the catalyst may be removed by filtration. The organic layer containing the product may be distilled to obtain the product as a residue, or it may be used in the next step directly.

Step d) involves reacting 1-(2-ethyl-5-fluoro-2,3,dihydro-1H-2-indenyl)-1-ethanone of Formula V with a brominating agent, to form 2-bromo-1-(2-ethyl-5-fluoro-2,3,dihydro-1H-2-indenyl)-1-ethanone of Formula VI.

Suitable brominating agents include liquid bromine, aqueous HBr, acetic acid HBr, N-bromosuccinamide (NBS), and the like.

Suitable solvents that may be used in the reaction of step d) include, but are not limited to, straight chain or branched alcohols, such as, for example, methanol, ethanol, isopropanol, butanol, and the like.

In an embodiment, 2-bromo-1-(2-ethyl-5-fluoro-2,3,dihydro-1H-2-indenyl)-1-ethanone is prepared using bromine and methanol at a temperature in the range of about 0-40° C. for about 2 to 6 hours, to minimize formation of a dibromo impurity.

Step e) involves converting 2-bromo-1-(2-ethyl-5-fluoro-2,3,dihydro-1H-2-indenyl)-1-ethanone into fipamezole or a salt thereof, using formamide, optionally in the presence of ammonia.

In an embodiment, the conversion of 2-bromo-1-(2-ethyl-5-fluoro-2,3,dihydro-1H-2-indenyl)-1-ethanone to fipamezole is carried out using formamide and ammonia.

In embodiments, suitable temperatures for the reaction range from about 70-180° C., or about 145-155° C., and the reaction is carried out for about 1 to 4 hours, or 1 to 2 hours. Longer times also can be used.

In a specific embodiment of the process, ammonia gas is sparged into a reaction mixture comprising 2-bromo-1-(2-ethyl-5-fluoro-2,3,dihydro-1H-2-indenyl)-1-ethanone and formamide at 150-155° C., for about 1 to 2 hours.

After completion of the reaction, the reaction mixture is cooled to 0-10° C. and pH may be adjusted to about 2-3, using concentrated HCl. The reaction mixture is washed with a water immiscible solvent, such as dichloromethane. The aqueous layer is made basic with aqueous ammonia and the product is extracted with an organic solvent, such as ethyl acetate. The organic layer may be concentrated to obtain a residue, or it may be used directly in the next step.

Fipamezole that is obtained may be converted into a pharmaceutically acceptable salt by reaction with an acid in a suitable solvent, such as, for example, acetone, methyl ethyl ketone, methyl isobutyl ketone, methanol, ethanol, isopropanol, and the like.

Pharmaceutically acceptable acids include hydrochloric acid, hydrobromic acid; sulphuric acid, phosphoric acid, acetic acid, and many others.

In this multi-step process, each step is contemplated separately or in combinations of any two or more steps, for the preparation of fipamezole.

In an aspect, the present application also provides processes for purifying fipamezole or a salt thereof, embodiments comprising:

a) providing a mixture of fipamezole or a salt thereof in an alcohol solvent;

b) combining the solution with a ketone anti-solvent and then cooling the mixture to form a precipitate; and

c) recovering purified fipamezole or a salt thereof.

The step of providing a solution includes dissolving fipamezole or a salt thereof in an alcohol solvent, or obtaining a solution from a step in which fipemazole is synthesized. Any forms of fipamezole or a salt thereof, such as crystalline forms, amorphous form, or mixtures of amorphous and crystalline forms in any proportions, obtained by any method, are acceptable for forming the solution.

Suitable alcohol solvents that may be used include C₁₋₆ straight chain or branched alcohols, such as, for example, methanol, ethanol, isopropanol, butanol, and the like.

Suitable ketone anti-solvents include acetone, methyl ethyl ketone, methyl isobutyl ketone, and the like.

In embodiments, the mixtures of fipamezole and alcohol are provided at the reflux temperature of the alcohol solvent used. The ketone anti-solvent may be added at reflux or at a lower temperature. Alternatively, the solution can be added to a ketone anti-solvent, at any temperatures up to the boiling point. The solution is then cooled to temperatures ranging about 0-30° C., or 20-25° C.

The method by which a solid material is collected from the final mixture can be any of techniques such as decantation, filtration by gravity or by suction, centrifugation, and the like. The solid so isolated can carry a small proportion of occluded mother liquor. If desired, the solid can be washed with a suitable solvent or mixtures of solvents in various proportions to wash out the mother liquor.

Drying can be suitably carried out using equipment such as a tray dryer, vacuum oven, air oven, fluidized bed dryer, spin flash dryer, flash dryer and the like. The drying may be carried out at temperatures about 35° C. to about 70° C. The drying can be carried out for any time periods necessary for obtaining a desired purity, such as from about 1 to about 25 hours, or longer.

In embodiments, there is provided fipamezole or a salt thereof having high purity, containing less than about 0.5%, or less than about 0.1%, by weight of process-related impurities, as characterized using techniques such as high performance liquid chromatography (“HPLC”).

In aspects, the present application provides the intermediate compounds 1-(5-fluoro-1-oxo-2,3-dihydro-1H-2-indenyl)-1-ethanone of Formula III, and 1-(2-ethyl-5-fluoro-1-oxo-2,3,dihydro-1H-2-indenyl)-1-ethanone of Formula IV, and their uses in the preparation of fipamezole or a salt thereof.

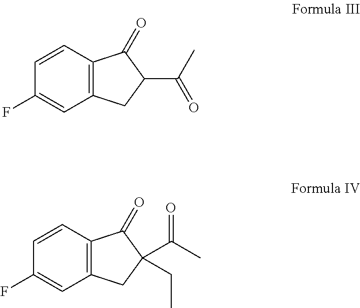

In an aspect, the present application also includes pharmaceutical formulations comprising fipamezole or a pharmaceutically acceptable salt thereof, prepared by a process of the present application, together with one or more pharmaceutically acceptable excipients.

Certain specific aspects and embodiments of this disclosure are described in the examples below, which are provided only for the purpose of illustration and are not intended to limit the scope of the disclosure in any manner.

### EXAMPLE 1

### Preparation of 5-fluoro-1-indanone (Formula II)

### A) Preparation of 3-chloro-1-(4-fluorophenyl)-propan-1-one

AlCl₃ (9.02 g) and dichloromethane (DCM, 50 mL) are charged into around bottomed flask under a nitrogen atmosphere. Chloropropionyl chloride (7.93 g) is added over 20 minutes at 25-30° C. Fluorobenzene (5 g) is added over about 15 minutes at 30° C. The mixture is maintained at 25-30° C. for about 12 hours. After reaction completion, water (50 mL) is added slowly and the mixture is stirred for 15 minutes. The aqueous layer is separated and extracted with DCM (50 mL). The combined organic layers are dried with sodium sulphate and distilled under vacuum below 50° C. n-Hexane (15 mL) is added to the residue and stirred for 20 minutes. The solid is filtered and washed with 10 mL of n-hexane (10 mL). The wet solid is dried, to obtain 6.9 g of 3-chloro-1-(4-fluorophenyl)-propan-1-one.

### B) Preparation of 5-fluoro-1-indanone (Formula II)

3-Chloro-1-(4-fluorophenyl)-propan-1-one (10 g) and concentrated sulfuric acid (60 mL) are charged into a 250 mL round bottom flask equipped with nitrogen gas flow and heated to 120° C. The mixture is stirred for about 30 minutes at 115-120° C. Reaction completion is confirmed using thin layer chromatography (TLC). The reaction mixture is poured into ice water (30 mL) followed by stirring for about 15 minutes. The mass is extracted with chloroform (150 mL) and the organic layer is concentrated under reduced pressure below 50° C. The obtained residue is purified by column chromatography using silica gel and n-hexane/ethyl acetate as a gradient eluent, to obtain 3.7 g of the title compound.

### EXAMPLE 2

### Preparation of 2-acetyl-5-fluoroindan-1-one (Formula III)

Into a 250 mL round bottom flask equipped with nitrogen gas flow are placed sodium hydride (1.6 g, 60% dispersion in mineral oil), THF (15 mL), 5-fluoro-1-indanone (3 g), and ethyl acetate (3.52 g), and the mixture is stirred at 30° C. for about 15 minutes. The mixture is heated to 50° C. and maintained for about 3 hours under a nitrogen atmosphere. After reaction completion, the mixture is cooled to 30° C., ice water (40 mL) is added, and pH is adjusted to about 5 using 10% aqueous HCl (4 mL). The mixture is extracted with ethyl acetate (90 mL) and the organic layer is concentrated under reduced pressure below 55° C. Isopropanol (15 mL) is added to the residue and stirred for about 1 hour at 30° C. The solid is filtered and washed with isopropanol (15 mL), to obtain 2.3 g of the title compound as a solid.

¹HNMR (CDCl₃, 400 MHz): 2.16(s, 3H), 3.52(s, 2H), 7.0-7.89(3H, m, ArH), 14.52-14.90 (s, CH—OH).

Mass: M+1 (M.Wt.: 193).

### EXAMPLE 3

### Preparation of 2-acetyl-2-ethyl-5-fluoroindan-1-one (Formula IV)

Into a 500 mL round bottom flask equipped with nitrogen gas flow, potassium carbonate (18.03 g), DMF (125 mL), and 2-acetyl-5-fluoro-indan-1-one (25 g) are charged and stirred for about 10 minutes at 25-35° C. The mixture is heated to 50-55° C. and maintained for about 1 hour, to form an off-white anion complex. Ethyl iodide (24.5 g) is added and the mixture is maintained for about 6 hours at 50-55° C. After reaction completion, the mixture is cooled to 25-35° C. The mixture is filtered to remove solid material. Water (250 mL) is added to the filtrate and pH is adjusted to about 3 with concentrated HCl (25 mL). The product is extracted with toluene (375 mL) and the organic layer is washed with water (125 mL). The organic layer is concentrated under reduced pressure below 55° C. The residue obtained is purified by column chromatography, using silica gel and 3% ethyl acetate in n-hexane as the mobile phase, to obtain 21 g of the title compound.

Purity by GC: 98.6%.

¹HNMR (CDCl₃, 400 MHz): 0.830(t, 3H), 1.805(q, 2H), 2.188(s, 1H), 2.83-2.93 (2H, dd, the indan ring H2-1 or H2-3), 6.80-7.08 (3H, m, ArH).

Mass: M+1 (M.Wt.: 220).

### EXAMPLE 4

### Preparation of 2-(2-ethyl-5-fluoroindan-2-yl)-ethanone (Formula V)

Palladium on charcoal (5% by weight, 5 g), ethanol (100 mL), acetic acid (50 mL), and 2-acetyl-2-ethyl-5-fluoro-indan-1-one (5 g) are charged into a stainless steel vessel at 25-35° C. The mixture is maintained at 50-60° C. under 340-415 kPa (50-60 psi) hydrogen pressure for about 6 hours. Reaction completion is confirmed using TLC. The mixture is filtered and the filtrate is concentrated under reduced pressure below 50° C., to obtain 4.6 g of the title compound.

¹HNMR (CDCL₃, 400 MHz): 0.830(t, 3H), 1.805(q, 2H), 2.188(s, 1H), 2.83-2.93 (2H, dd, the indan ring H2-1 or H2-3), 3.35-3.46 (2H, dd, the indan ring H2-1 or H2-3), 6.80-7.02-7 (3H, m, Ar—H).

Mass: M+1 (M.Wt.: 206).

### EXAMPLE 5

### Preparation of 2-bromo-1-(2-ethyl-5-fluoroindan-2-yl) ethanone (Formula VI)

Methanol (30 mL) and 2-(2-ethyl-5-fluoroindan-2-yl)-ethanone (5 g) are charged into a 250 mL round bottom flask equipped with nitrogen gas flow and the mixture is cooled to −10° C. to 0° C. A mixture of bromine (3.9 g) and methanol (12.5 mL) is slowly added at −5° C. to 0° C. The mixture is stirred for about 4 hours. After completion of the reaction, water (25 mL) and sodium sulfite (1.25 g) are added and the mixture is allowed to reach 30° C. and stirred for about 10 minutes. The mixture is extracted with toluene (37.5 mL) and the organic layer is washed with aqueous sodium bicarbonate solution (10%, 25 mL). The organic layer is concentrated under reduced pressure below 55° C. The residue obtained is purified by column chromatography using silica gel and ethyl acetate/n-Hexane as a mobile phase, to obtain 4.2 g of the title compound.

### EXAMPLE 6

### Preparation of fipamezole hydrochloride

Formamide (12 mL) and 2-bromo-1-(2-ethyl-5-fluoroindan-2-yl) ethanone (4 g) are charged into a 100 mL round bottom flask equipped with nitrogen gas flow and the mixture is heated to about 150° C. The mixture is stirred for about 3 hours and then ammonia gas is sparged into the reaction mixture for about 2 hours at 150-155° C. After completion of the reaction, the mixture is cooled to 0-5° C. and water (2.0 mL) is added. The pH is adjusted to about 2 using concentrated HCl (4 mL). The mixture is allowed to reach 25-35° C. and is washed with dichloromethane (8 mL). The aqueous layer pH is adjusted to about 9-10 using aqueous ammonia solution (25%, 8 mL) and the aqueous layer is extracted with ethyl acetate (40 mL). The organic layer is separated and washed with water (20 mL) and aqueous sodium chloride solution (10%, 50 mL). The organic layer is concentrated under reduced pressure below 55° C., to obtain 3.1 g of residue.

Hydrogen chloride in ethanol (7% w/v, 31 mL) is added to the residue and stirred for about 1 hour at 25-30° C. The mixture is concentrated under reduced pressure below 55° C., to obtain 3.2 g of fipamezole HCl. Purity by HPLC: 98.94%.

### EXAMPLE 7

### Purification of fipamezole hydrochloride

Isopropyl alcohol (9.6 mL) and fipamezole HCl from Example 6 (3.2 g) are charged into a 100 mL round bottom flask equipped with nitrogen gas flow and the mixture is heated to reflux. The mixture is stirred for about 40 minutes and then acetone (32 mL) is added at reflux temperature. The mixture is then cooled to 25-30° C. and stirring is continued for about 6 hours. The formed solid is filtered and washed with cold acetone. The wet solid is dried, to produce 2.1 g of purified fipamezole HCl as a white solid.

Purity by HPLC: 99.8%.

### EXAMPLE 8

### Preparation of 5-fluoro-1-indanone (Formula II)

### A) Preparation of 3-fluorocinnamic acid

Malonic acid (15 g), pyridine (35 mL), and 4-fluorobenzaldehyde (10 g) are charged into a 500 mL round bottom flask equipped with nitrogen gas flow at 25° C. Piperidine (0.6 g) is added and the mixture is stirred for 10 minutes at 25-35° C. The mixture is heated to 110° C. and stirred for about 5 hours at 110-115° C. After completion of the reaction, the mixture is cooled to 25° C. and the reaction is quenched by adding a mixture of ice water (210 mL) and concentrated HCl (56 mL). The mixture is stirred for about 15 minutes. The obtained solid is filtered and washed with water (20 mL). The wet solid is charged into a flask containing a mixture of water (132 mL) and methanol (66 mL) and heated to 60° C. The mixture is stirred for about 30 minutes and then cooled to 25-35° C. The solid is filtered and dried below 50° C., to obtain 12 g of 3-fluorocinnamic acid as a solid.

### B) Preparation of 3-(3-fluorophenyl)propanoic acid

Palladium on charcoal (5% w/w, 2.5 g), methanol (50 mL), THF (50 mL), and 3-fluorocinnamic acid (10 g) are charged into a stainless steel vessel at 25-35° C. The mixture is maintained at 25-35° C. under 415-485 kPa (60-70 psi) hydrogen pressure for about 4 hours. Reaction completion is confirmed using TLC. The mixture is filtered and the solid washed with THF (25 mL). The filtrate is concentrated under reduced pressure below 50° C., to obtain 10 g of 3-(3-fluorophenyl)propanoic acid.

### C) Preparation of 5-fluoro-1-indanone (Formula II)

Polyphosphoric acid (200 g) is charged into a 500 mL round bottom flask equipped with nitrogen gas flow and heated to about 90° C. 3-(3-Fluorophenyl)propanoic acid (10 g) is added at 90° C. and the mixture is further heated to 110° C. The mixture is stirred for about 30 minutes at 100-110° C. After completion of the reaction, the mixture is cooled to 0-5° C. and cold water (1000 mL) is added. The mixture is extracted with ethyl acetate (200 mL). The organic layer is separated and washed with aqueous sodium bicarbonate solution (10%, 100 mL). The organic layer is concentrated under reduced pressure below 55° C., to obtain 8.5 g of the title compound as a solid.

1. A process for preparing fipamezole or a salt thereof, comprising:
a) reacting 1-(2-ethyl-5-fluoro-1-oxo-2,3,dihydro-1H-2-indenyl)-1-ethanone of Formula IV with a reducing reagent, to form 1-(2-ethyl-5-fluoro-2,3,dihydro-1H-2-indenyl)-1-ethanone of Formula V; 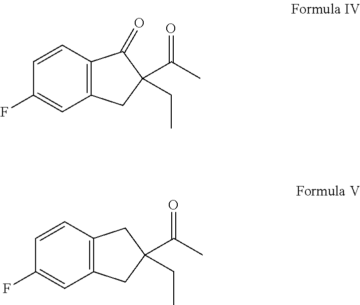
b) reacting 1-(2-ethyl-5-fluoro-2,3,dihydro-1H-2-indenyl)-1-ethanone of Formula V with a brominating agent, to form 2-bromo-1-(2-ethyl-5-fluoro-2,3,dihydro-1H-2-indenyl)-1-ethanone of Formula VI; and 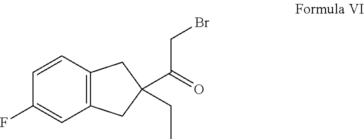
c) converting 2-bromo-1-(2-ethyl-5-fluoro-2,3,dihydro-1H-2-indenyl)-1-ethanone into fipamezole or a salt thereof.

2. The process of claim 1, wherein a reducing agent comprises a metal catalyst in combination with hydrogen.

3. The process of claim 2, wherein a metal catalyst comprises nickel, platinum, palladium, iridium and ruthenium.

4. The process of claim 2, wherein a metal catalyst is present on a solid inert support.

5. The process of claim 1, wherein the reaction of step a) is carried out in the presence of an acid comprising hydrochloric acid, hydrobromic acid, sulphuric acid, phosphoric acid, or acetic acid.

6. The process of claim 1, wherein a brominating agent comprises liquid bromine, aqueous HBr, acetic acid HBr, or NBS.

7. The process of claim 1, wherein the reaction of b) is carried out in a solvent comprising a C₁-C₆ straight chain or branched alcohol.

8. The process of claim 1, wherein the reaction of b) is carried out at temperatures in the range of about 0-40° C., for about 2 to 6 hours.

9. The process of claim 1, wherein converting of c) comprises reacting with formamide, optionally in the presence of ammonia.

10. The process of claim 1, further comprising reacting fipamezole with a pharmaceutically acceptable acid, to form a salt.

11. The process claim 10, wherein a pharmaceutically acceptable acid comprises hydrochloric acid, hydrobromic acid, sulphuric acid, phosphoric acid, or acetic acid.

12. A process for the preparation of 1-(2-ethyl-5-fluoro-1-oxo-2,3,dihydro-1H-2-indenyl)-1-ethanone of Formula IV, comprising: 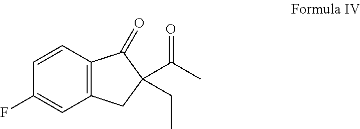
a) reacting 5-fluoro-1-indanone of Formula II with a lower alkyl ester reagent, in the presence of a base, to form 1-(5-fluoro-1-oxo-2,3-dihydro-1H-2-indenyl)-1-ethanone of Formula III: and 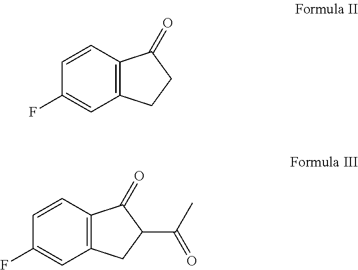
b) reacting 1-(5-fluoro-1-oxo-2,3,dihydro-1H-2-indenyl)-1-ethanone of Formula III with an ethyl halide, in the presence of a base, to form 1-(2-ethyl-5-fluoro-1-oxo-2,3,dihydro-1H-2-indenyl)-1-ethanone of Formula IV.

13. The process of claim 12, wherein a lower alkyl ester reagent comprises ethyl acetate, n-propyl acetate, isopropyl acetate, n-butyl acetate, or t-butyl acetate.

14. The process of claim 12, wherein a base in a) comprises sodium hydride, potassium hydride, sodium methoxide, or sodium amide.

15. The process of claim 12, wherein the reaction of step a) is carried out in a solvent comprising tetrahydrofuran, toluene, or xylene.

16. The process of claim 12, wherein an ethyl halide comprises ethyl fluoride, ethyl chloride, ethyl bromide, or ethyl iodide.

17. The process of claim 12, wherein a base used in b) comprises an alkali metal hydroxide, alkaline metal hydroxide, alkali metal carbonate, alkaline earth metal carbonate, or alkali metal bicarbonate.

18. The process of claim 12, wherein the reaction of b) is carried out in a solvent comprising a polar aprotic solvent, tetrahydrofuran, a hydrocarbon, a halogenated hydrocarbon, and any mixtures thereof.

19. A process for purifying fipamezole or a salt thereof, comprising:
a) providing a solution of fipamezole or a salt thereof in an alcohol solvent;
b) combining the solution with a ketone anti-solvent and cooling to form a precipitate; and
c) recovering purified fipamezole or a salt thereof.

20. The process of claim 19, wherein an alcohol solvent comprises a C₁₋₆ straight chain or branched alcohol.

21. The process of claim 19, wherein an alcohol solvent comprises methanol, ethanol, isopropanol, or butanol.

22. The process of claim 19, wherein a ketone anti-solvent comprises acetone, methyl ethyl ketone, or methyl isobutyl ketone.

23. The process of claim 19, wherein a purified fipamezole or salt thereof contains less than about 0.5% by weight of process-related impurities.

24. A compound 1-(5-fluoro-1-oxo-2,3-dihydro-1H-2-indenyl)-1-ethanone of Formula III.

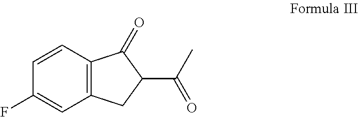

25. A compound 1-(2-ethyl-5-fluoro-1-oxo-2,3,dihydro-1H-2-indenyl)-1-ethanone of Formula IV. [chemical structure]
